(19) United States
(12) Patent Application Publication
(10) Pub. No.: US 2011/0257585 A1
(43) Pub. Date: Oct. 20, 2011

(54) Medical Apparatus

(75) Inventors: Chris Althorpe, Cardiff (GB); Andrew McNeill, Anstruther Fife (GB)

(73) Assignee: AMBICARE HEALTH LIMITED, EDINBURGH (GB)

(21) Appl. No.: 13/132,122

(22) PCT Filed: Dec. 1, 2009
(86) PCT No.: PCT/GB09/51629

§ 371 Date: Jul. 12, 2011

(30) Foreign Application Priority Data
Dec. 1, 2008 (GB) 0821818.2

(51) Int. Cl. A61M 37/00; A61N 5/06

(52) U.S. Cl. 604/20; 607/90

(57) ABSTRACT

There is herein described medical apparatus. More particularly, there is herein described medical apparatus capable of adapting and/or conforming to a non-planar surface on a patient's body.

### FIELD OF THE INVENTION

The present invention relates to medical apparatus. More particularly, the present invention relates to medical apparatus capable of adapting and/or conforming to a non-planar surface on a patient's body.

### BACKGROUND OF THE INVENTION

Traditional light sources are typically rigid structures and are not flexible. Moreover, these types of traditional light sources contain electronics which further adds to their rigidity. Therefore, in applications where a flat light source is required to conform to a neighbouring surface, this lack of flex limits the degree of conformability. Traditional light sources that do flex have been found to be prone to breaking (or disconnecting from electronics) when folded past a certain angle.

Light can be used to treat a wide variety of diseases. When light alone is used to treat a disease, the treatment is referred to as phototherapy. Light may be used in conjunction with a pharmaceutical in which case the treatment is called photodynamic therapy.

Phototherapy and photodynamic therapy can be used to treat a variety of skin and internal diseases. In photodynamic therapy, a light-sensitive therapeutic agent known as a photopharmaceutical is supplied externally or internally to an area of the body which is to be treated. That area is then exposed to light of a suitable frequency and intensity to activate the photopharmaceutical. A variety of photopharmaceutical agents are currently available. For example there are topical agents such as 5-aminolevulinic acid hydrochloride (Crawford Pharmaceuticals), methylaminolevulinic acid (Metfix (Trade Mark), Photocure). Often, the drug is applied in a non-active form that is metabolised to a light-sensitive photopharmaceutical.

In photodynamic therapy, the primary technique for supplying light to the photopharmaceutical is to project light of a suitable wavelength from standalone light sources such as lasers or filtered arc lamps. These sources are cumbersome and expensive, and are therefore only suitable for use in hospitals.

This leads to inconvenience for the patient, and high cost for the treatment. High light irradiances are needed in order to treat an acceptable number of patients per day (for the treatment to be cost effective) and to avoid unduly inconveniencing the patient.

It is an object of at least one aspect of the present invention to obviate or mitigate at least one or more of the aforementioned problems.

It is a further object of at least one aspect of the present invention to provide apparatus capable of providing therapeutic and/or cosmetic treatment and wherein the apparatus is capable of adapting and/or conforming to a non-planar surface on a patient's body.

It is a yet further object of at least one aspect of the present invention to provide medical apparatus comprising a light source and electronics wherein said medical apparatus is capable of adapting and/or conforming to a non-planar surface on a patient's body.

### SUMMARY OF THE INVENTION

According to a first aspect of the present invention there is provided an apparatus for use in therapeutic and/or cosmetic treatment, the apparatus comprising:

a casing; and

electronics located within or attached to the casing;

wherein the casing and electronics are capable of adapting and/or conforming to a non-planar surface.

The casing may restrict the amount of conformation of the electronics.

The apparatus may therefore be medical apparatus which may be used for a variety of therapeutic and/or cosmetic treatments.

The casing may also comprise, contain or be attached to a light source capable of distributing light over an area of a patient to be treated.

Typically, the casing may surround at least part or preferably all of the electronics and/or the light source.

The apparatus of the present invention may therefore provide a light source which is capable of adapting and/or conforming to a non-planar, substantially non-planar or curved surface on, for example, any part of a human or mammal body such as on a foot, leg, torso, shoulder, arm, hand, head or facial area of a patient.

By allowing the casing to adapt and/or conform to a non-planar or curved surface allows the light source to be capable of providing even or substantially even illumination of a patient's skin, which is important for efficacious therapy. The present invention may therefore allow a semi-rigid light source to adapt and/or conform to non-planar and/or curved surfaces on a patient.

The apparatus may therefore comprise means capable of allowing the apparatus to move from a first configuration where a surface(s) of the apparatus intended to contact the patient is planar or substantially planar to a second configuration wherein the surface(s) of the apparatus intended to contact the patient is non-planar, substantially non-planar and/or curved. The casing may there be adaptable and/or conformable from a first configuration to a second configuration wherein the second configuration may be non-planar, substantially non-planar and/or curved. The apparatus may therefore comprise means allowing the apparatus to bend, flex and/or conform around a non-planar, substantially non-planar and/or curved surface. The apparatus and/or the casing may therefore be bendable, flexible and/or conformable.

The casing may have means of restricting the amount of conformability that it is capable of achieving. This would prevent the electronics from being flexed to such a degree that they break or fail.

The casing may comprise a surface intended to make contact with a patient (i.e. a contact surface). This contact surface may be capable of bending, flexing and/or conforming around a non-planar, substantially non-planar or curved surface of, for example, a patient.

The casing may be capable of bending, flexing and/or conforming away from a planar position by less than about 5 degrees, less than about 10 degrees, less than about 15 degrees, less than about 20 degrees, less than about 25 degrees, less than about 30 degrees, less than about 35 degrees, less than about 40 degrees, less than about 45 degrees, less than about 50 degrees, less than about 55 degrees, less than about 60 degrees, less than about 65 degrees, less than about 70 degrees, less than about 75 degrees, less than about 85 degrees or less than about 90 degrees. Alternatively, the casing may be capable of bending, flexing and/or conforming away from a planar position by about 0-5 degrees, about 0-10 degrees, about 0-15 degrees, about 0-20 degrees, about 0-25 degrees, about 0-30 degrees, about 0-35 degrees, about 0-40 degrees, about 0-45 degrees, about 0-50 degrees, about 0-55 degrees, about 0-65 degrees, about 0-70 degrees, about 0-75 degrees, about 0-80 degrees, about 0-85 degrees or about 0-90 degrees. As indicated above the casing may comprise a surface intended to make contact with a patient (i.e. a contact surface) and it may be this surface which may be capable of bending, flexing and/or conforming away from a planar position.

The casing may also comprise means that once the casing is flexed, bent and/or conformed from an initial configuration (e.g. planar), this means urges the casing back to the initial configuration, for example, by a spring-type arrangement.

In a particular embodiment, the casing may comprise two sections, for example, an upper section and a lower section. Alternatively, there may be a plurality of sections. Typically, the casing may be in any appropriate shape such as in a disk-like form. The two sections of the casing may be attached to one another during use using any suitable mechanical and/or adhesive means. For example, the two sections of the casing may be attached to one another using attachment means such as a snap-fit arrangement, clips, hooks, Velcro (Trade Mark) and the like. In at least one or both of the two sections of the casing, there may be means allowing the casing to bend, flex and/or conform. Typically, there may be at least one or two flexible portions in at least one or both of the two sections of the casing. In embodiments where there may be two flexible portions in each of the two sections of the casing, the flexible portions may be located substantially opposite one another on each of the sections of the casing. Preferably, each of the two sections comprises two flexible portions wherein each of the two flexible portions in the upper and lower sections are located substantially adjacent to one another in the casing. There may also be a plurality of flexible portions. Typically, the flexible portion may be a hinge. In particularly preferred embodiments, there may be at least two or a plurality of hinges on each of the sections of the casing.

Typically, the casing may also comprise means which may restrict the amount by which the casing or a surface intended to make contact with a patient is capable of bending, flexing and/or conforming away from a planar arrangement. The casing may therefore comprise a form of stop means capable of restricting the amount of movement allowed. An elongate member in the form of, for example, a tubular member such as a bar, rod, pipe may extend through the at least one or two of the means allowing the casing to bend, flex and/or conform and thereby restrict the amount of bending, flexing and/or conforming. For example, this may be achieved by the elongate member abutting and/or striking against part of, for example, the inner surface of the casing to prevent any further bending, flexing and/or conforming. By restricting the amount of bending, flexing and/or conforming reduces any possible damage to electronics and a light source located within the casing.

The casing may therefore comprise hinges which may be substantially ‘n’ shaped and may be made from a flexible material (e.g. a web of material). The ‘n’ shaped hinges made from, for example, flexible material, may be designed to limit the amount of bending, flexing and/or conforming that may occur by straightening or substantially straightening so that no further bending may be allowed. Alternatively, the hinge may be formed from any non-linear (i.e. not straight) and/or non-planar shape that may be straightened or substantially straightened and thereby extend the straight length of the hinge. The straightening of the non-linear and/or non-planar shape may therefore allow the casing to bend, flex and/or conform. Once the non-linear and/or non-planar shape is fully straightened no further bending, flexing and/or conforming may be allowed. For example, a zig-zag, helical and/or coil arrangement may be used. Alternatively, the hinge may be formed from any non-linear (i.e. not straight) and/or non-planar shape and also from an elastic material such that once the casing has been bent, flexed and/or conformed the hinge may act so as to straighten the casing back to its planar configuration.

The casing may comprise electronics for the operation of the apparatus and the light source. The casing may also be designed to allow light from the light source to exit from the casing and be used in medical applications such as therapeutic and/or cosmetic treatment. The casing may therefore be capable of distributing light over an area of a patient to be treated.

Typically, the casing may also comprise means such as protruding members, fingers, teeth, catch members and the like which are capable of retaining the electronics and the light source within the casing.

Typically, the casing comprises an annular member formed, for example, by the upper and lower sections. Within the perimeter of annular member the casing may comprise openings which facilitate bending, flexing and/or conforming. Conveniently, the centre of the casing may comprise an open structure which further facilitates bending, flexing and/or conforming. The surface of the casing intended to make contact with a patient may have a surface area of about 0.1-500 cm², about 0.1-250 cm², about 0.1-100 cm², about 1-100 cm² or about 5-50cm². Alternatively, the surface of the casing intended to make contact with a patient may have a surface area of greater than about 0.01 cm², greater than about 0.1 cm², greater than about 1 cm², greater than about 5 cm², greater than about 10 cm², greater than about 50 cm² or greater than about 100 cm².

Conveniently, the casing may be made from any suitable material such as any suitable plastics material, composite and/or metal based material. The material forming the casing may be pliable and may bend, flex and/or conform.

The casing may also comprise an adhesive sheet around at least part or the entire periphery of the casing. The adhesive sheet may provide an adhesive surface for attaching the apparatus to a patient. The combination of a casing capable of adapting to a non-planar and/or curved surface with a flexible adhesive periphery increases the number of sites that the apparatus may be attached to.

The apparatus may also comprise a drug and/or chemical source capable of delivering drugs and /or chemicals to the area of the patient to be treated. The apparatus of the present invention may therefore be capable of performing a therapeutic and/or cosmetic treatment.

The present invention may therefore relate to a light emitting apparatus capable of performing a therapeutic and/or cosmetic treatment on a human or animal patient using photodynamic therapy or phototherapy. In some situations the treatment may be said to be of therapeutic nature (e.g. skin cancer, serious acne) whereas in other situations the treatment may be said to be of cosmetic nature (e.g. minor amounts of acne, anti-aging treatments such as treatment of wrinkling).

The light source may be any suitable light source that may emit light over a desired wavelength. The light source may be provided as a light emitting layer. Typically, the light source may operate within a range of about 300-3000 nm, about 300-1500 nm, about 300-800 nm or about 370-700 nm. In particular embodiments the light source may operate in the visible region of the electromagnetic spectrum. Alternatively, the light source may operate in the ultraviolet or infrared wavelength regions of the electromagnetic spectrum. The light source during operation may operate at a substantially constant and/or uniform wavelength or alternatively may be scanned over a range of wavelengths. In particular embodiments more than one wavelength of light may be emitted and/or pulsed at the same or different times. This may be suitable for the treatment of acne.

The light source may have an extensive light emitting surface area of about 0.1-500 cm², about 0.1-250 cm², about 0.1-100 cm², about 1-100 cm² or about 5-50 cm². Alternatively, the light source may have a light emitting surface of greater than about 0.01 cm², greater than about 0.1 cm², greater than about 1 cm², greater than about 5 cm², greater than about 10 cm², greater than about 50 cm² or greater than about 100 cm².

The light source may be substantially square or substantially rectangular and may have dimensions of about 0.5 cm×0.5 cm, about 1 cm×1 cm, about 2 cm×2 cm, about 5 cm×5 cm, about 10 cm×10 cm or about 20 cm×10 cm.

Alternatively, the light source may be substantially circular and may have a diameter of about 0.5 cm, about 1 cm, about 2 cm, about 5 cm, about 10 cm or about 20 cm.

The light source may emit light substantially continuously over a pre-set period of time or may emit light discontinuously such as in a pulsed manner. In particular embodiments, the light from the light source may be pulsed with a period of at least about 10 ms, at least about 100 ms, at least about 1 s, at least about 10 s, at least about 10 ms, at least about 100 s, at least about 1,000 s or at least about 10,000 s.

In particular embodiments, the light source may, for example, be any suitable form of diode such as organic light-emitting diode or an inorganic light-emitting diode incorporated into, for example, a diffuser. Alternatively, the light source may be a fluorescent light source such as a fluorescent lamp. The fluorescent light source may be compact in shape. For example, this includes technology developed from back-lit display technology, LEDs and waveguides and diffusers.

The light source may have an optical power density of about 0.1-500 mW/cm², about 1-200 mW/cm² or about 5-50 mW/cm². The light source during operation may operate at a substantially constant power or alternatively may be varied over a range of powers.

The apparatus may comprise a photochemical and/or a photopharmaceutical preparation in the drug and/or chemical source in the form of, for example a layer.

The photochemical and/or photopharmaceutical preparation may be delivered to the area of the patient to be treated at appropriate pre-set times and/or may be controlled by a control unit. The photochemical and/or a photopharmaceutical preparation may be present in the form of, for example, a gel, ointment, cream or gauze soaked in a photodynamic therapy solution. Alternatively, or in addition the light emitting device may be provided with a thin film impregnated with a photochemical and/or photopharmaceutical preparation.

The photochemical and/or a photopharmaceutical preparation may comprise a drug and/or antiseptic capable of treating a patient which may be applied to a person in need thereof. Typically, the photochemical and/or photopharmaceutical may be transparent or substantially transparent or may become transparent or substantially transparent during use and emission of light.

In embodiments where the photochemical and/or photopharmaceutical may be transparent or translucent to the wavelength of the irradiating light, the resulting device may be readily applied without a separate step of applying a photochemical and/or a photopharmaceutical to a patient. In particular embodiments, the photochemical and/or photopharmaceutical may be covered with a peelable release medium. The photochemical and/or photopharmaceutical preparation may comprise an inactive compound which may be metabolised in vivo to an active compound. During use a suitable amount of the photochemical and/or photopharmaceutical preparation may be applied to an area of a patient to be treated.

Typical drugs and/or chemicals used in the present invention include but are not limited to precursor drugs such as ALA or Metfix or other photodynamic therapy agents.

In use, the drug and/or chemical layer may be placed over the top of the area of the patient to be treated. To facilitate treatment an electrolyte solution such as sodium chloride solution may be placed on the skin to improve the electrical contact.

A power source may be supplied to power the light source and/or the electronics within the casing. The power source may be small and compact and may be integrated into the whole apparatus therefore making the apparatus suitable for ambulatory treatment. The apparatus of the present invention may therefore be wearable and may be attached to a foot, leg, torso, shoulder, arm, hand, head or facial area of a patient.

The apparatus may comprise attachment means for attaching the device to a body part(s) of a human or animal. For example, the apparatus may comprise mechanical and/or adhesive means for attaching the device to a body part of a human or animal. In particular embodiments, the apparatus may therefore comprise a strap arrangement which may optionally comprise a fastening means and/or Velcro (Trade Mark) and/or an adhesive surface for attaching the apparatus to the patient.

The apparatus may be lightweight and portable. In particular embodiments the apparatus may be a totally self-contained portable unit and may comprise a self-contained power supply. The power supply may operate electronics in the device and the light source. The apparatus may be sufficiently portable to enable ambulatory treatment therefore allowing treatment during which a patient may move around. Treatment may therefore occur at home or at work and may be removed by a patient when necessary. This provides lower treatment costs as this avoids out-patient or in-patient stays in hospital. This provides the significant advantage that lower light levels may be used since exposure can occur for a longer period of time. This overcomes the problem of pain induced in some patients by high irradiances from conventional sources used in hospitals. Moreover, lower irradiance over a longer period of time may be more effective in photoinduced therapy as it gives more time for oxygen to diffuse to the region to be treated, and reduces photobleaching of the photopharmaceutical.

The light source may also comprise a substrate layer such as a transparent or at least a substantially transparent substrate layer. Alternatively, the light source may comprise a translucent or at least a substantially translucent substrate layer. The substrate layer may function as a support layer for the light source and may allow the light to penetrate there through. The substrate layer may also function as a barrier layer too and may be selected to prevent oxygen and/or moisture from penetrating the light source. The substrate layer may be made from or comprise any one of or combination of suitable materials such as a glass, plastics or polymer. Additional layers may also be present.

The apparatus according to the present invention may be used in a range of phototherapies and photodynamic therapies. For example, the apparatus according to the present invention may be used in the treatment of cancer (e.g. skin cancer), acne, wrinkles, wound-healing, anti-aging and post-skin laser treatments such as found in cosmetic applications. By wound is meant any form of open or closed wound. Open wounds include but are not limited to: incisions or incised wounds; lacerations; abrasions; puncture wounds; penetration wounds; gunshot wounds; and ulcers (including diabetic derived ulcers). Closed wounds include but are not limited to: contusions; hemaomas; and crushing injuries.

According to a second aspect of the present invention there is provided a method of performing a therapeutic and/or cosmetic treatment, the method comprising:

providing a light source capable of distributing light over an area of a patient to be treated;

providing a casing surrounding at least part of the light source;

wherein the casing and electronics are capable of adapting and/or conforming to a non-planar surface.

The method may be performed by an apparatus as defined in the first aspect.

The casing may restrict the amount of conformation of the electronics. According to a third aspect of the present invention there is provided use of an apparatus according to the first aspect in a medical treatment.

Typically, the medical treatment may be a photodynamic therapy or phototherapy.

The present invention may therefore relate to the treatment of cancer (e.g. skin cancer), acne, wrinkles, wound-healing, anti-aging and post-skin laser treatments (e.g. cosmetic applications).

According to a fourth aspect of the present invention there is provided a method of medical treatment using of an apparatus according to the first aspect.

The medical treatment may be that of photodynamic therapy or phototherapy.

Moreover, the treatment may be that of the treatment of cancer (e.g. skin cancer), acne, wrinkles, wound-healing, anti-aging and post-skin laser treatments (e.g. cosmetic applications).

### BRIEF DESCRIPTION OF THE DRAWINGS

Embodiments of the present invention will now be described, by way of example only, with reference to the accompanying drawings in which:

FIG. 1 is a representation of an apparatus according to an embodiment of the present invention;

FIG. 2 is an enlarged view of part of the apparatus shown in FIG. 1; and

FIG. 3 is a representation of an apparatus according to a further embodiment of the present invention.

### BRIEF DESCRIPTION

The present invention therefore relates to an apparatus capable of performing a therapeutic and/or cosmetic treatment on a human or animal patient. The apparatus may be used for any type of medical treatment and, for example, may be used in phototherapy or photodynamic therapy.

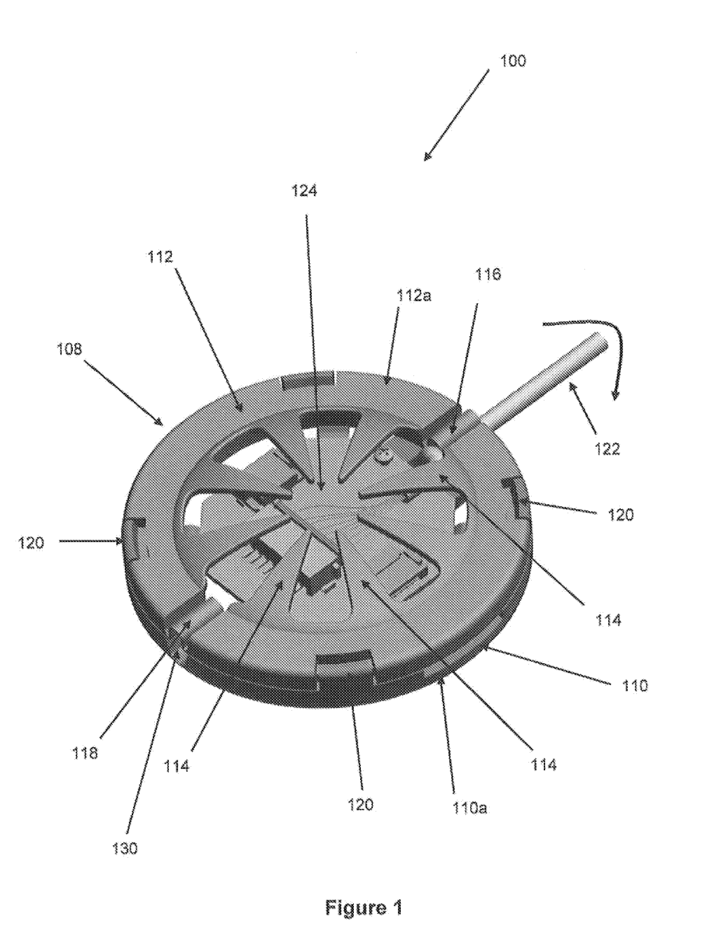

FIG. 1 is a representation of an apparatus according to the present invention, generally designated 100. The apparatus 100 comprises a casing 108 made from, for example, plastics material. The casing 108 is of a substantially disc shape and comprises a lower section 110 and an upper section 112. As shown in FIG. 1, the upper section 112 comprises a series of members 114 which may be used to hold or protect electronics 124 and a light source (not shown) within the casing 108. The lower section 110 and upper section 112 comprise annular members 110a112a, respectively. FIG. 1 also shows that within the annular members 110a, 112a, the device 108 contains a series of gaps between the members 114 which facilitates bending, flexing and/or conforming. The centre of the casing 108 also comprises an open structure which further facilitates bending, flexing and/or conforming.

The lower section 110 and the upper section 112 are connected to one another with a snap-fit arrangement such as clips 120 extending from the lower section 110 on to an upper portion of the upper section 112.

On the upper section 112 there are two hinge members 116,118 that allow some flexibility in the casing 108. The casing may therefore bend, flex and/or conform to a part of a patient's body. The hinges are substantially ‘n’ shaped and may be made from a flexible material (e.g. a web of material). The flexible material is designed to limit the amount of bending, flexing and/or conforming that may occur by straightening so that no further bending is allowed.

Furthermore, as shown in FIG. 1 a tubular member 122 extends through hinge member 116.

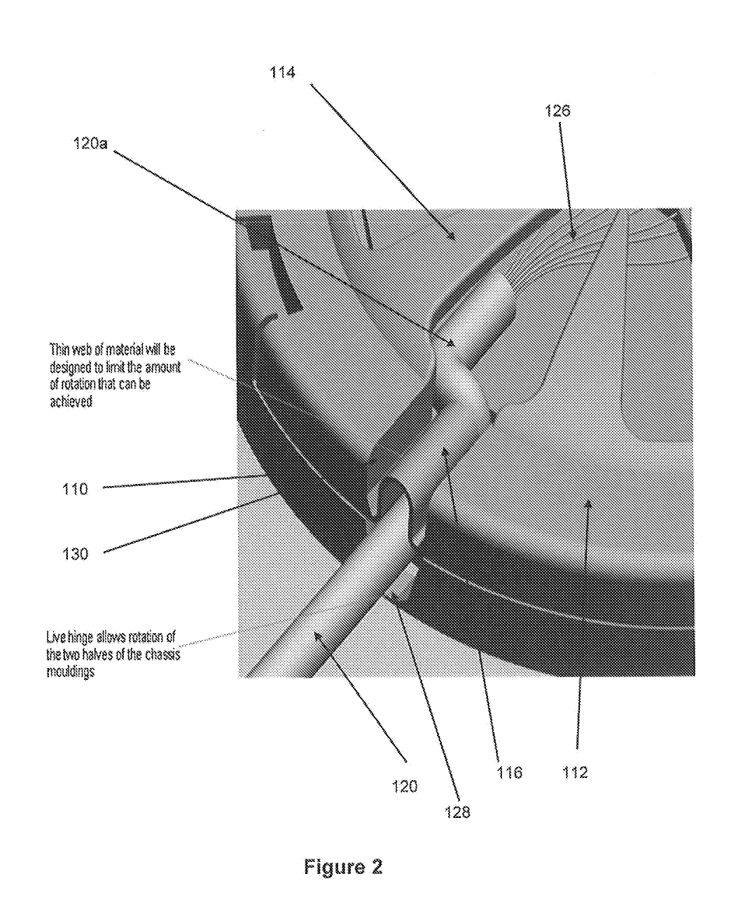

FIG. 2 is an expanded view of the hinge 116 and the tubular member 122 extending therethrough. FIG. 2 also shows a hinge 128 in the lower section 110 of the casing 100. A similar hinge 130 is also shown in FIG. 1 in the lower section 110. The hinge 128,130 are flexible and allow the casing 108 to bend, flex and/or conform to a part of a patient's body. The hinges 116,118 and hinges 128,130 are directly or substantially directly opposite one another in the casing 108. When the casing 108 flexes on hinges 130 and 128 the folded hinges 116 and 118 are straightened. Once they have been straightened the casing will not bend any further.

The tubular member 122 comprises a section 120a which is bent away from the rest of the tubular member 120. The bent tubular section 120a is capable of restricting the amount by which the casing 108 may bend, flex and/or conform. In use, the bent section 120 abuts and/or strikes against an underside portion of one of the members 114 which are used to hold the electronics in place.

The tubular member 122 as shown in FIG. 2 is also used to contain wires 126 connecting to the electronics 124 located in the casing 108.

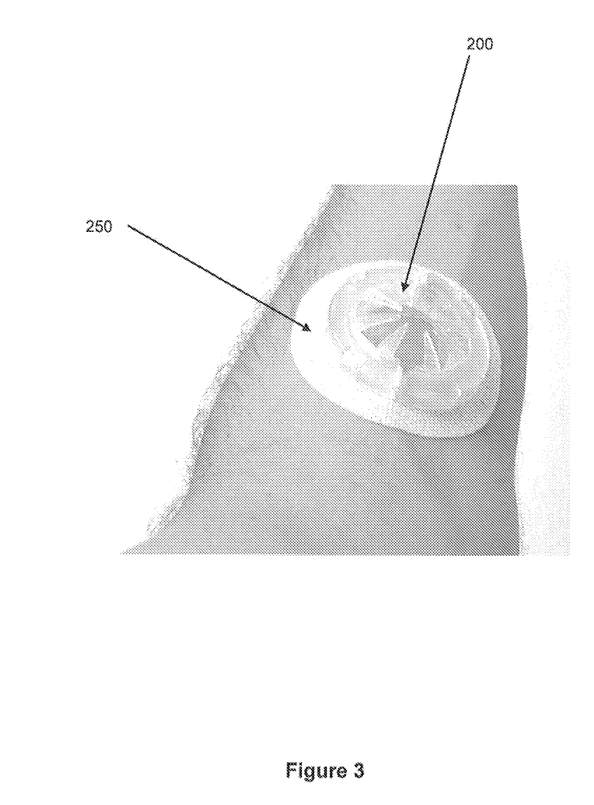

FIG. 3 is a representation of a further apparatus according to the present invention, generally designated 200. The apparatus 200 is similar in many regards to the apparatus 100 shown FIGS. 1 and 2 but has a flexible adhesive sheet 250 around the periphery of the apparatus 200. This flexible sheet 250 provides an adhesive surface for attaching the apparatus 200 to a patient. The combination of a hinged apparatus 200 with a flexible periphery increases the number of sites that can be attached to, when compared to the flexible apparatus 200 alone.

Whilst specific embodiments of the present invention have been described above, it will be appreciated that departures from the described embodiments may still fall within the scope of the present invention. For example, any suitable type of hinge mechanism may be used to allow the casing to bend, flex and/or conform to a part of a patient's body. Moreover, any suitable type of light source may be used. Furthermore any suitable type of drug and/or antiseptic may be delivered using apparatus according to the present invention.

1-25. (canceled)

26. A light emitting apparatus for use in therapeutic and/or cosmetic treatment, the apparatus comprising:
a casing; and
electronics located within or attached to the casing;
wherein the casing and electronics are capable of adapting and/or conforming to a non-planar surface.

27. The apparatus of claim 26, wherein the casing also comprises, contains or is attached to a light source capable of distributing light over an area of a patient to be treated, and wherein the casing surrounds at least part or all of the electronics and/or the light source.

28. The apparatus of claim 26, wherein the apparatus is capable of adapting and/or conforming to a foot, leg, torso, shoulder, arm, hand, head or facial area of a patient; wherein the casing is capable of adapting to a non-planar surface and capable of allowing a light source to provide even or substantially even illumination of a patients skin, and wherein the casing is adaptable and/or conformable from a first configuration to a second configuration wherein the second configuration is non-planar, substantially non-planar and/or curved.

29. The apparatus of claim 26, wherein the apparatus comprises means capable of allowing the apparatus to move from a first configuration where a surface(s) of the apparatus intended to contact the patient is planar or substantially planar to a second configuration wherein the surface(s) of the apparatus intended to contact the patient is non-planar, substantially non-planar and/or curved, wherein the apparatus comprises means allowing the apparatus to bend, flex and/or conform around a non-planar, substantially non-planar and/or curved surface, wherein the casing comprises a surface intended to make contact with a patient (i.e. a contact surface) and this contact surface is capable of bending, flexing and/or conforming around a non-planar, substantially non-planar or curved surface of a patient, wherein the casing is capable of bending, flexing and/or conforming away from a planar position by less than about 5 degrees, less than about 10 degrees, less than about 15 degrees, less than about 20 degrees, less than about 25 degrees, less than about 30 degrees, less than about 35 degrees, less than about 40 degrees, less than about 45 degrees, less than about 50 degrees, less than about 55 degrees, less than about 60 degrees, less than about 65 degrees, less than about 70 degrees, less than about 75 degrees, less than about 85 degrees or less than about 90 degrees; or wherein the casing is capable of bending, flexing and/or conforming away from a planar position by about 0-5 degrees, about 0-10 degrees, about 0-15 degrees, about 0-20 degrees, about 0-25 degrees, about 0-30 degrees, about 0-35 degrees, 0-40 degrees, about 0-45 degrees, 0-50 degrees, about 0-55 degrees, about 0-65 degrees, about 0-70 degrees, about 0-75 degrees, about 0-80 degrees, about 0-85 degrees or about 0-90 degrees.

30. The apparatus of claim 26, wherein the casing further comprises means which when the casing is flexed, bent and/or conformed from an initial configuration (e.g. planar) urges the casing back to the initial configuration, and wherein the casing comprises an upper section and a lower section and the upper and lower section are attached to one another using mechanical and/or adhesive means, and wherein both of the two section of the casing comprise means allowing the casing to bend, flex and/or conform.

31. The apparatus of claim 26, wherein the casing is capable of adapting and/or conforming to a non-planar surface using a hinge or a plurality of hinges, and wherein the hinge or the plurality of hinges are formed from substantially ‘n’ shaped members made from or comprising a flexible material wherein the flexible material is designed to limit the amount of bending, flexing and/or conforming that may occur by straightening so that no further bending is allowed.

32. The apparatus of claim 26, wherein the casing comprises means capable of restricting the amount by which the casing or a surface intended to make contact with a patient is capable of bending, flexing and/or conforming, and wherein the casing comprises a stop means capable of restricting the amount of movement allowed, and wherein the stop means is in the form of an elongate member which extends through at least one or a plurality of means allowing the casing to bend, flex and/or conform and thereby restrict the amount of bending, flexing and/or conforming by abutting and/or striking against part of the casing.

33. The apparatus of claim 26, wherein a surface of the casing intended to make contact with a patient has a surface area of about 0.1-500 cm² , about 0.1-250 cm² about 0.1-100 cm², about 1-100 cm² or about 5-50 cm²; or wherein a surface of the casing intended to make contact with a patient has a surface area of greater than about 0.01 cm², greater than about 0.1 cm², greater than about 1 cm², greater than about 5 cm², greater than about 10 cm², greater than about 50 cm² or greater than about 100 cm².

34. The apparatus of claim 26, wherein the casing is made from or comprises a plastics material, composite and/or metal based material.

35. The apparatus of claim 26, wherein the casing comprises an adhesive sheet around at least part or all of the periphery of the casing.

36. The apparatus of claim 26, wherein the apparatus also comprises a drug and/or chemical source capable of delivering drugs and/or chemicals to the area of the patient to be treated.

37. The apparatus of claim 26, wherein the apparatus comprises a light source capable of operating within a range of about 300-3000 nm, about 300-1500 nm, about 300-800 nm or about 370-700 nm.

39. The apparatus of claim 26, wherein the apparatus is a totally self-contained portable unit.

40. A method of performing a therapeutic and/or cosmetic treatment, the method comprising:
providing a light source capable of distributing light over an area of a patient to be treated; and
providing a casing surrounding at least part of the light source;
wherein the casing and electronics are capable of adapting and/or conforming to a non-planar surface, and wherein the treatment is photodynamic therapy or phototherapy.
